(19) United States
(12) Patent Application Publication
(10) Pub. No.: US 2026/0273119 A1
(43) Pub. Date: Sep. 17, 2026

(54) METHOD FOR INTRODUCING ITEMS FROM A CONTAINER INTO THE PROCESS CHAMBER OF A CONTAINMENT UNDER ASPECTIC CONDITIONS, AND LOCK ARRANGEMENT THEREFOR

(71) Applicant: SKAN AG, Allschwil (CH)

(72) Inventors: Julia Annette Carli, Basel (CH); Philipp LEUENBERGER, Basel (CH); Mathieu MÜLLER, Sierentz (FR); Christophe RETHORET, Issenheim (FR); Tobias Jan SCHNEIDLER, Hagendorn (CH); Marc TONDERA, Riehen (CH); Mike ZELLER, Wahlen (CH)

(73) Assignee: SKAN AG, Allschwil (CH)

(21) Appl. No.: 18/873,830

(22) PCT Filed: May 25, 2023
(86) PCT No.: PCT/CH2023/000003

§ 371 Date: Dec. 11, 2024

(30) Foreign Application Priority Data
Jul. 26, 2022 (EP) 22187101.5

(51) Int. Cl. A61L 2/24; A61L 2/10; A61L 2/208

(52) U.S. Cl. CPC A61L 2/24; A61L 2/10; A61L 2/208; A61L 2202/121; A61L 2202/122; A61L 2202/123

(57) ABSTRACT

A method and to a lock arrangement for the introduction of articles (960) under aseptic conditions from containers (9;91,92,93), which have been supplied in series, into a process chamber (43), which is surrounded by a housing (49), of a containment (4) by a lock arrangement (1) positioned in a setup area (A). An input station (3) is upstream of the process chamber (43). A single container (9;91,92,93) has a vessel (94) with an aseptic interior space (940) and an aperture (95), which has been closed with a cover (98). The articles (960) which have been stored in the interior space (940) are to be treated in the process chamber (43) after the cover (98) is opened.

### CROSS-REFERENCE TO RELATED APPLICATIONS

This application is a 371 National Phase of PCT/CH2023/000003, filed May 25, 2023, which claims priority to European Patent Application No. 22187101.5, filed Jul. 26, 2022, both of which are incorporated herein by reference as if fully set forth.

### TECHNICAL FIELD

The present invention relates to a method and to a lock arrangement which is positioned in a setup area and intended for introducing articles under aseptic conditions from containers, which have been supplied in series, into a process chamber, which is surrounded by a housing, of a containment. An input station is upstream of the process chamber. A single container comprises a vessel with an aseptic interior space and an aperture, which has been closed with a cover. The articles stored in the interior space are to be treated in the process chamber after the cover is opened. The vessels of the containers have the form of trough-shaped tubs, and the articles are e.g. pharmaceutical ampullae, vials or syringes, which fit into systematically arranged, depression-like receiving contours of nests.

### BACKGROUND

### Prior Art

WO 2020/016 645 A2 discloses an arrangement for the contamination-free introduction of a sterile object from a vessel, which has been closed with a semipermeable cover, into a working chamber, which is surrounded by a wall, of a containment. The arrangement comprises first of all a portal unit having an access flange which is arranged in the wall and forms a passage from the outside into the working chamber, and a door which sealingly closes the passage and, in order to be opened, can be moved into the working chamber. The arrangement also comprises a vessel receptacle having a repository for holding a vessel that has been introduced into the vessel receptacle, an opening for the introduction of the vessel into the repository, and a flange for interacting with the access flange.

A decontamination unit is provided to, when the door has been closed, the vessel receptacle has been docked to the access flange and the vessel has been stored in the vessel receptacle, decontaminate an outer surface, facing toward the door, of the cover. In the closed position, the door is attached in a sealed manner to the access flange from the side of the working chamber, and the access flange borders an intermediate space which is situated in the passage. The cover of the vessel which has been placed in the vessel receptacle is sealed between the flange and the access flange when the vessel receptacle has been pivoted into abutment against the access flange. The decontamination unit, with a direction of action into the passage, is installed directly on the door or laterally with respect to the portal unit that has the door and the access flange. The decontamination unit is in the form of a radiation source, e.g. UVC, UV, E-beam or pulsed light, or in the form of a fumigation device, e.g. for atomizing an H₂O₂ solution.

### OBJECT AND SUMMARY OF THE INVENTION

In terms of the limited productivity of the existing devices and implemented methods according to the already known prior art, the invention is based on the object of proposing a more efficient method for the field of use in question for introducing articles from a container into the process chamber of a containment under aseptic conditions and a lock arrangement suitable therefor.

What is critical here is to make the degree of automation as high as possible, extensively avoid manual intervention, and ensure high cleanliness requirements. Moreover, the priority needs to be transferring, to the greatest possible extent, spending as short as possible a decontamination time and with minimum outlay on decontamination equipment, only articles that are to be processed into the process chamber.

The method according to the invention, or the lock arrangement according to the invention, is designed for the introduction of articles under aseptic conditions from containers, which have been supplied in series, into a process chamber, which is surrounded by a housing, of a containment by means of a lock arrangement positioned in a setup area. An input station is upstream of the process chamber. A single container comprises a vessel with an aseptic interior space and an aperture, which has been closed with a cover, the articles stored in the interior space being intended for treatment in the process chamber after the cover has been opened.

In the production mode of the lock arrangement, the following method steps are carried out in succession:
to transfer the articles from the containers into the process chamber through from the input station at least two passages leading into the process chamber are provided and each of the passages is closed on one side by a lid that has been guided into abutment in the process chamber and/or on the other side by the cover, facing toward the passage, of a container that has been guided into abutment in the input station;
when a container has been guided into abutment with the passage, that surface of the cover that faces toward the passage, together with an enclosed surrounding area, is decontaminated by means of a decontamination device;
after that surface of the cover that faces toward the passage, including the surrounding area thereof, has been decontaminated, the cover is opened, in order to transfer the articles from the vessel through its aperture into the process chamber; and,
after the vessel is emptied, firstly the lid is placed back onto the respective passage in order to then remove the vessel, from which the articles have been discharged, from the passage; wherein
the lid is used on the passages alternately.

Special embodiments of the method and lock arrangement according to the invention are defined below:
The decontamination device is integrated in the lid or arranged outside of the lid, the emission element of the decontamination device being fitted directly in the lid or outside of the lid in a receptacle.

The enclosed surrounding area includes the gas volume it contains and surfaces around which the gas volume flows, i.e. surfaces:
of the lid;
of the optional receptacle;
of an optional seal; and
of optionally exposed wall areas.

The process chamber is equipped with a manipulator or, additionally, with a second manipulator, fitted on the pivotable extension arms of which in each case is a tool head provided with tools, wherein:
the tools are designed to open the cover and then transfer the articles from the vessel through the opened-up aperture into the process chamber; and
the one manipulator with its tools is used interchangeably on the passages to open the cover and transfer the articles into the process chamber; or
the one manipulator and the second manipulator are each used only on one of the passages, or the two manipulators are used interchangeably on the passages.

The tools include a cutting tool, in order to cut open the cover for the opening operation. With preference, at least parts of the area of the cover and of the enclosure consist of a semipermeable nonwoven fabric, such as Tyvek®. The cover, before it is removed, keeps the interior space of the vessel in a sterile state and is sealed, e.g. by adhesive bonding or welding, on an edge that runs around the aperture of the vessel.

Stored in the vessel for the purpose of receiving a multiplicity of articles is a removable nest which, together with the articles, is transferred into the process chamber. After the articles have been discharged from the nest in the process chamber, this empty nest is cleared from the process chamber into an empty vessel which comes next or which is the same one and has been pressed against the respective passage. As an alternative, the articles are transferred directly from the nest that remains in the vessel into the process chamber.

The decontamination device is preferably in the form of a radiation source, e.g. UVC, UV, E-beam or pulsed light, or alternatively in the form of a fumigation device, e.g. for atomizing an H₂O₂ solution.

The input station, which is surrounded by a housing, has an access point leading to an adjacent providing station, wherein:
the input station is operated with a cleaned, unidirectional displacement flow, some of which exits through the access point;
at the providing station, the containers that, in the as-supplied state, are each surrounded by an enclosure in order to keep the internal volume of the container, together with its contents, aseptic are supplied; and
at the providing station, an opening, which has been made on the enclosure and until now was kept closed, is then opened up on the side facing toward the displacement flow exiting the access point, in order to introduce the single container into the input station; or
at the providing station, the enclosure is separated on the side facing toward the displacement flow exiting the access point, in order to introduce the single container into the input station.

The container fitted in the initially still closed enclosure is positioned on a support. A feeding means is used to push the container out of the open enclosure, when there is a displacement flow flowing onto the container. The enclosure is held by a gripper, preferably in the form of a suction device.

The containers that have been introduced into the input station are fed to the passages by means of at least one conveying device and taken away again after the processing via this conveying device. The at least one conveying device is preferably based on the principle of magnetic levitation and allows conveying on multiple tracks, and also in opposite directions.

A first conveying device is used to transport the containers within the input station, while a second conveying device causes the containers to be pivoted into abutment with their cover pressed against the respective passage. The second conveying device can also be used to remove processed containers from the respective passage. To position the containers against the respective passage, the second conveying device preferably comprises a cardanic bearing.

The second conveying device consists of:
a shaft, through which a first axis of rotation extends;
two forks, which are fixed next to one another on the shaft and are each pivotable about a second axis of rotation extending at right angles to the first axis of rotation; and
a respective carrier which is suspended in the associated fork and can be pivoted about a third axis of rotation, wherein the first axis of rotation and the third axis of rotation run parallel to one another and the carriers are configured to grip the edges of the vessels from underneath while the vessels are being pivoted onto and off of the passages.

Two cardanic bearings that are next to one another are each formed from a U-shaped carrier, which is connected in the third axis of rotation in each case to a fork, which is suspended in the second axis of rotation.

Fitted in each passage is a seal which, together with the cover of a container which has been temporarily pressed against and/or together with a lid which has been temporarily pressed against the passages, provides sealing in the transition from the input station to the process chamber. Instead of a conventional seal, it is possible to provide a minimal gap or use a labyrinth seal. The lid is carried by a pivotable extension arm of an apparatus positioned in the process chamber.

The at least one manipulator or the additional manipulator is preferably in the form of a robot.

The vessels of the containers have the form of trough-shaped tubs. The articles are e.g. ampullae, vials or syringes, which fit into systematically arranged receiving contours of nests.

The essence of the invention is based on the method and the product having one or more of the features disclosed herein.

### BRIEF DESCRIPTION OF THE DRAWINGS

In the drawings:

FIG. 1A—shows a container which is to be processed by the method according to the invention in the associated lock arrangement and has a closed enclosure;

FIG. 1B—shows an exploded view of the container as per FIG. 1A without the enclosure;

FIG. 2A—shows a perspective view of the second conveying device as a constituent part of the lock arrangement, consisting of the shaft and two cardanic bearings fixed thereto;

FIG. 2B—shows a plan view of a cardanic bearing from FIG. 2A;

FIGS. 3A to 15D: show the entirety of a method sequence, with respectively each group of four figures showing the lock arrangement, specifically in front section as per Figures A, in rear section as per Figures B, in plan view as per Figures C and in a main side view as per Figures D;

FIGS. 3A to 3D—show process phase 1 (start position);

FIGS. 4A to 4D—show process phase 2;

FIGS. 5A to 5D—show process phase 3;

FIGS. 6A to 6D—show process phase 4;

FIGS. 7A to 7D—show process phase 5;

FIGS. 8A to 8D—show process phase 6;

FIGS. 9A to 9D—show process phase 7;

FIGS. 10A to 10D—show process phase 8;

FIGS. 11A to 11D—show process phase 9;

FIGS. 12A to 12D—show process phase 10;

FIGS. 13A to 13D—show process phase 11;

FIGS. 14A to 14D—show process phase 12;

FIGS. 15A to 15D—show process phase 13;

FIG. 16A—shows an alternative arrangement with the decontamination device outside of the lid, fitted in a mount, the container pressed against the passage by means of the pivoted-up second conveying device, the lid closed, and the decontamination device activated; and

FIG. 16B—shows the arrangement as per FIG. 16A with an open lid and an inactive decontamination device.

### DETAILED DESCRIPTION

The method according to the invention and the lock arrangement according to the invention for introducing articles from a container into the process chamber of a containment under aseptic conditions will be described in detail below with reference to the appended drawings. Like in the appended drawings, the embodiment described below relates to the transfer of articles from containers into the process chamber through two passages leading from the input station into the process chamber. Each of the passages can be closed alternately by a lid which has been guided into abutment in the process chamber and in which the emission element of a decontamination device can be integrated.

For the entirety of the description that follows, the following statement applies: where reference numerals are included in a figure for the purposes of clarity of the drawing and it is clear from the drawing that a “repeat” component is involved, but are not discussed in the directly associated text of the description, for the sake of brevity reference is made to the explanation thereof in preceding figure descriptions.

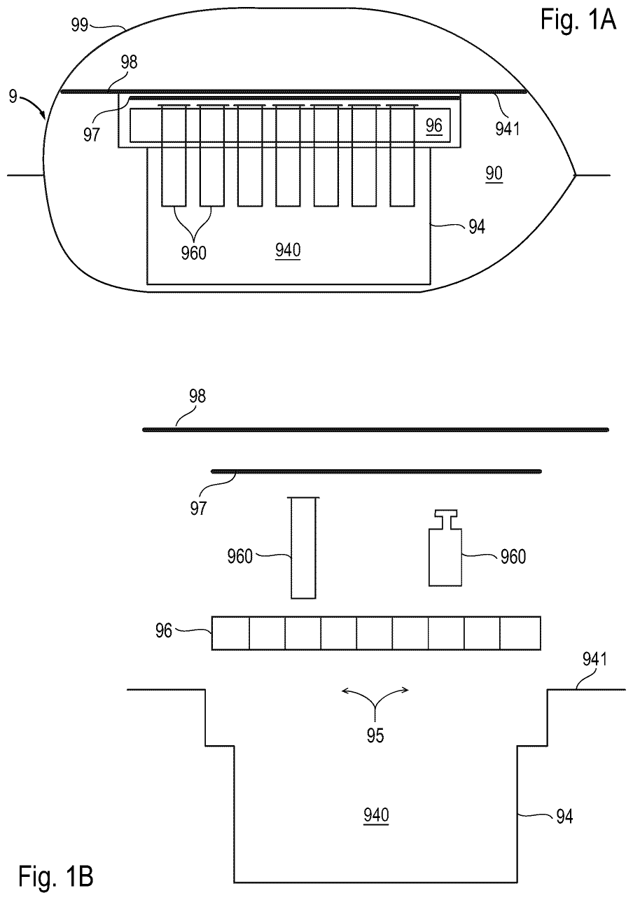

### FIGS. 1A and 1B

A single container 9 comprises:
a vessel 94 with an aseptic interior space 940 and an aperture 95, which has been closed with a cover 98;
a nest 96, which is stored in the interior space 940 and fitted into the trough-shaped receiving contours of which are the articles 960 that, after the cover 98 is opened, are to be treated in the process chamber 43 of the containment 4;
optionally an intermediate layer 97 inserted between the cover 98 and the nest 96, above the articles 960; and
a closed enclosure 99 which surrounds the entire container 9 in the as-supplied state and keeps the internal volume 90 sterile.

The vessel 94 is e.g. a trough-shaped tub. The articles 960 are in particular ampullae, vials or syringes. With preference, at least parts of the area of the cover 98 and of the enclosure 99 consist of a semipermeable nonwoven fabric, such as Tyvek®. The cover 98 is typically sealed on the edge 941 that runs around the aperture 95 of the vessel 94 and thus keeps the interior space 940 of the vessel 94 in a sterile state.

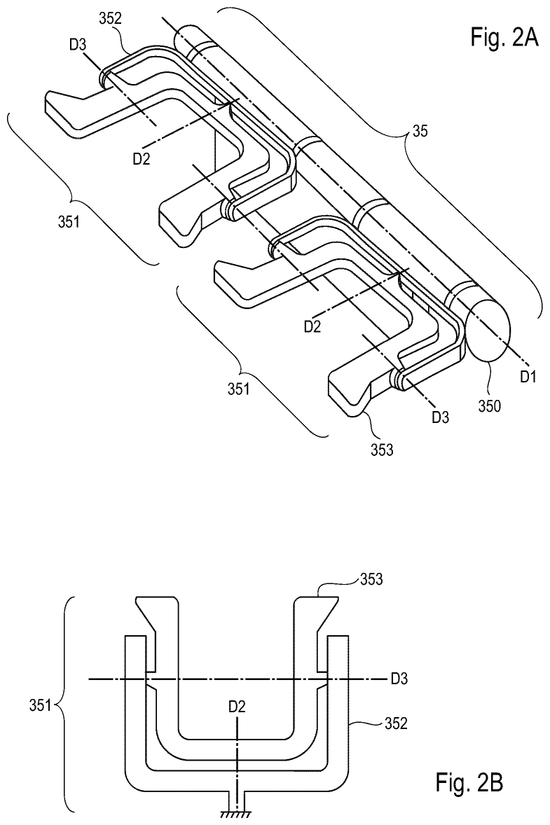

### FIGS. 2A and 2B

As constituent part of the lock arrangement 1, the front first zone 31 and the rear second zone 32 of the input station 3 are each equipped with a second conveying device 35. Such a conveying device 35 consists of a shaft 350 and two cardanic bearings 351 fixed thereto. A first axis of rotation D1 extends through the shaft 350. Two forks 352, which are fixed next to one another on the shaft 350 and are each pivotable about a second axis of rotation D2 extending at right angles to the first axis of rotation D1. A carrier 353 which can be pivoted about a third axis of rotation D3 is suspended in each fork 352, wherein the first axis of rotation D1 and the third axis of rotation D3 run parallel to one another. The carriers 353 are configured to grip the edges 941 of the vessels 94 from underneath while the vessels are being pivoted onto and off of the passages 47,48 that lead from the input station 3 into the process chamber 43.

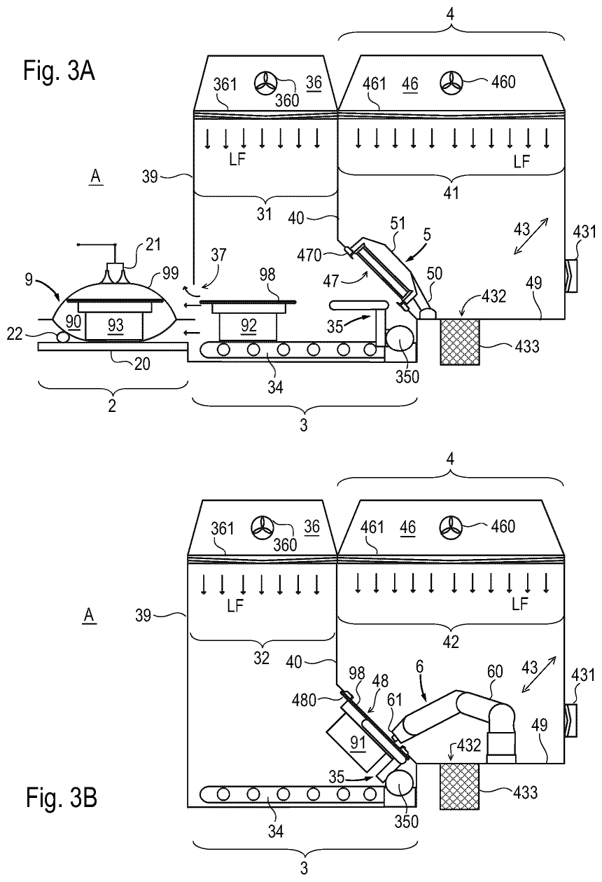

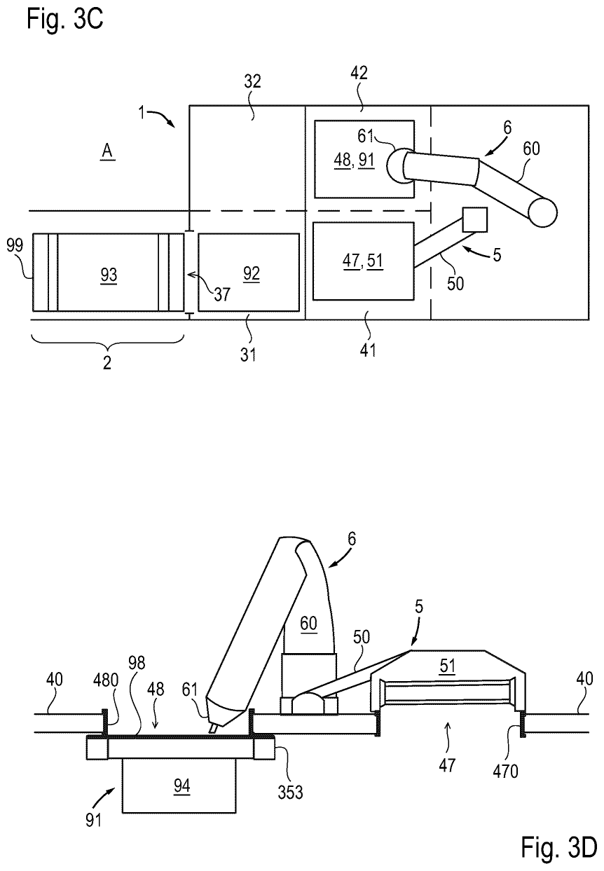

### FIGS. 3A to 3D

### Structure of the Lock Arrangement 1

The lock arrangement 1 installed in the setup area A is made up of an input station 3, a providing station 2 upstream thereof, and the containment 4, with its process chamber 43, that adjoins the input station 3. The providing station 2 has the support 20, in order to position thereon new, third containers 93 with an initially still closed enclosure 99. The feeding means 22 is used to push those third containers 93 out of the opened enclosure 99 and off of the support 20 through the access point 37 in the housing 39, which surrounds the input station 3, onto the first conveying device 34 in the front first zone 31 of the input station 3. During the pushing-out operation, the gripper 21 which is in the providing station 2 retains the enclosure 99. The second conveying device 35 adjoins the first conveying device 34. Arranged in the plenum 36 of the front first zone 31 of the input station 3 are a supply-air fan 360 and a supply-air filter 361 for generating a cleaned air flow LF that flows laminarly downstream.

Between the front first zone 31 of the input station 3 and the front first zone 41 of the process chamber 43 extends the partition 40 which comprises the first passage 47 between the zones 31,41. The seal 470 is fitted in the first passage 47. Also fitted in the plenum 46 of the front first zone 41 of the containment 4 are a supply-air fan 460 and a supply-air filter 461 for generating a cleaned air flow LF that flows laminarly downstream. Provided in the housing 49 that surrounds the process chamber 43 are, laterally and/or alternatively in its floor, an exhaust-air filter 431 and, in the floor, a waste opening 432, which leads to a repository 433.

Stationed in the common process chamber 43 that extends via the front first zone 41 and the rear second zone 42 is an apparatus 5 having the extension arm 50 and the lid 51, which is held thereon and in which the decontamination device 7 is integrated. By pivoting the extension arm 50, it is possible to guide the lid 51, via intermediate positions, into abutment with the first passage 47 or the second passage 48.

In the input station 3, the rear second zone 32 is behind the first zone 31, and in the containment 4, the rear second zone 42 is behind the first zone 41. Equivalently to the first zone 31, the second zone 32, likewise in its plenum 36, is equipped with a supply-air fan 360 and a supply-air filter 361 for generating an air flow LF. Present in turn are a first conveying device 34 and the second conveying device 35 that adjoins the first. Similarly equivalently to the first zone 41, the second zone 42 comprises the plenum 46 with a supply-air fan 460 and a supply-air filter 461 for generating the air flow LF.

In smaller installations, a common supply-air fan 360 and a common supply-air filter 361 for the first zone 31 and the second zone 32 and also a common supply-air fan 460 and a common supply-air filter 461 for the first zone 41 and the second zone 42 may be sufficient. In larger installations, multiple supply-air fans 360,460 and supply-air filters 361,461 are provided for the zones 31,32 and 41,42, respectively. The rear second zone 42 also contains in the housing 49, laterally and/or alternatively in its floor, the exhaust-air filter 431 and, in the floor, the waste opening 432, which leads to a repository 433.

Between the rear second zone 32 of the input station 3 and the rear second zone 42 of the process chamber 43 extends afresh the partition 40, which then comprises the second passage 48 between the zones 32,42. The seal 480 is fitted in this second passage 48. Stationed in the process chamber 43, which is common to the first and second zone 41,42, is a manipulator 6 having the extension arm 60 and the tool head 61 thereon. By pivoting the extension arm 60, it is possible to guide the tool head 61, via intermediate positions, into abutment with the first passage 47 or the second passage 48.

### Sequence in Process Phase 1 (Start Position)

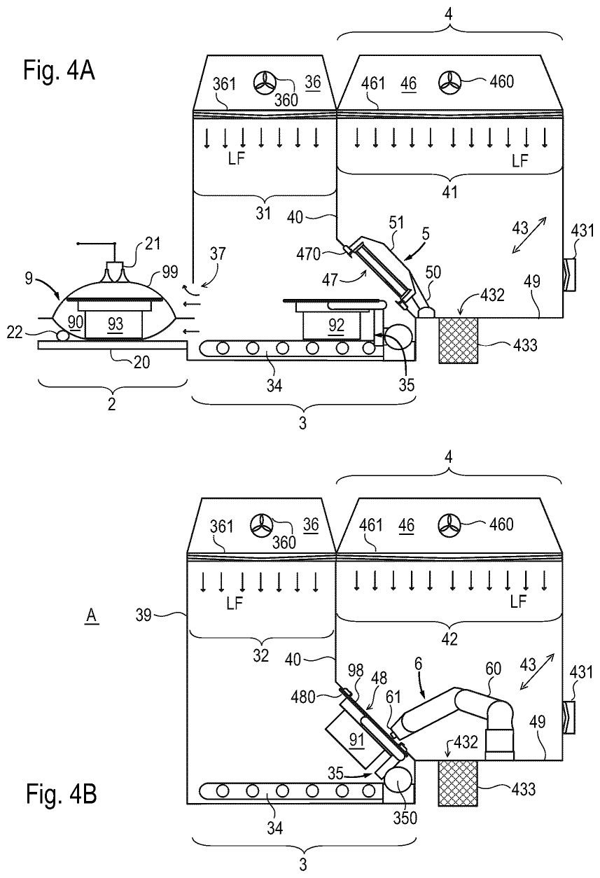

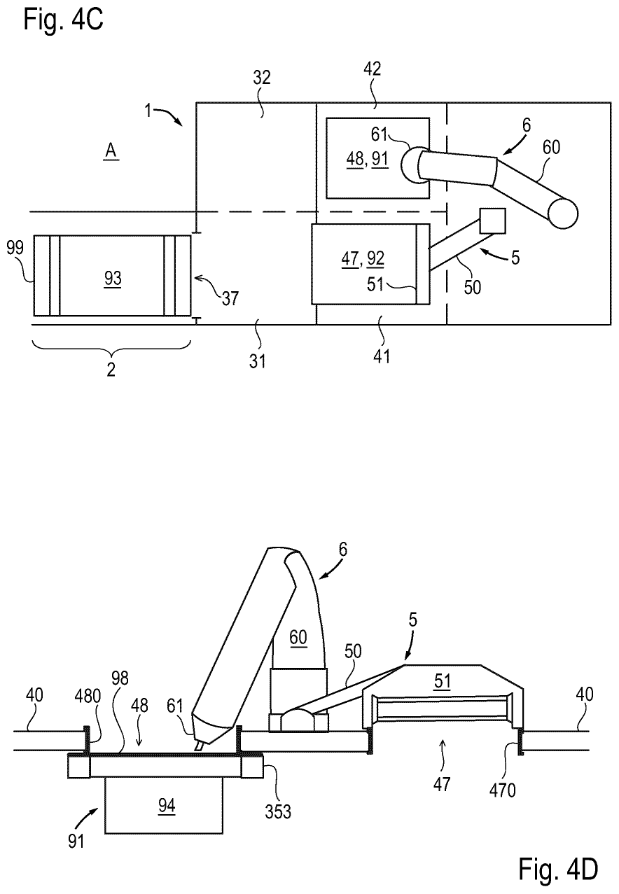

In the providing station 2:
A new, third container 93, with its enclosure 99 still closed, is supplied on the support 20, is situated in front of the access point 37 and is exposed to the flow LF that flows out through the latter.
In the input station 3, in its front first zone 31:
A second container 92, already freed from the enclosure 99, is on the first conveying device 34. With respect to the first zone 31, the first passage 47 is open, as it were.
In the input station 3, rear second zone 32:
The second passage 48 into the process chamber 43 of the containment 4 is closed by the cover 98 of the first container 91, which has been pressed thereagainst, but already decontaminated.
In the containment 4, front first zone 41:
The first passage 47 into the process chamber 43 is closed by the lid 51 having the decontamination device 7 integrated therein. The lid 51 was guided into abutment by the apparatus 5 with the pivoted extension arm 50 out of the process chamber 43.
In the containment 4, rear second zone 42:
A tool, which is mounted on the tool head 61 of a pivoted extension arm 60 of the manipulator 6 that is stationed in the process chamber 43, is used to open, e.g. cut open, and remove the cover 98.
In the following description of the sequence in the further process phases 2 to 13 (FIGS. 4A to 15D), to avoid repetitions in each case only the modifications in relation to the previous process phase are specified.

### Sequence in Process Phase 2 (FIGS. 4A to 4D)

In the input station 3, front first zone 31:
The second container 92, without an enclosure 99 and still standing on the first conveying device 34, is taken up by the second conveying device 35.

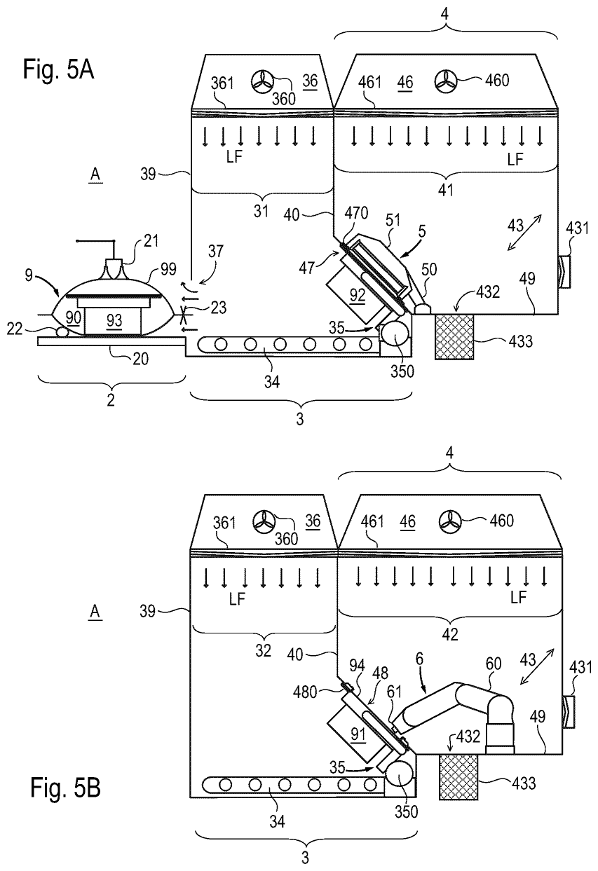

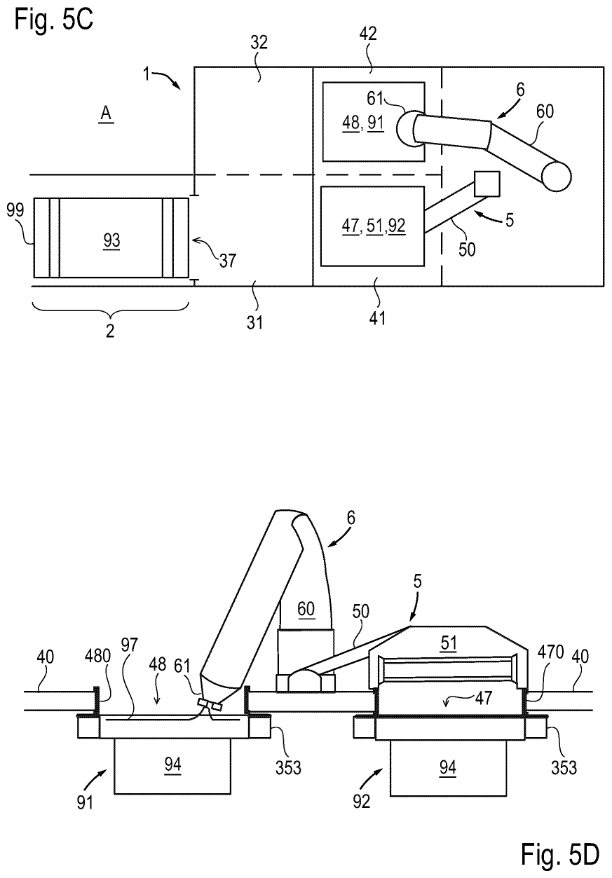

### Sequence in Process Phase 3 (FIGS. 5A to 5D)

In the providing station 2:
The until-now closed enclosure 99 around the new, third container 93 is opened, e.g. cut open, by means of the tool 23; in the process, the gripper 21 holds the enclosure 99. As an alternative, a cut, which has been made on the enclosure 99 beforehand and is initially clamped shut, can now be opened up.
In the input station 3, front first zone 31:
The second conveying device 35 has guided the second container 92 into abutment with the second passage 48, which is then closed, one on each side, by the second container 92, which has been pressed thereagainst, and the lid 51, which has been placed thereon.
In the containment 4, rear second zone 42:
A tool, which is installed on the tool head 61 of the pivotable arm 60 of the manipulator 6, removes the optionally present interlayer 97 from the first container 91 when the cover 98 is open.

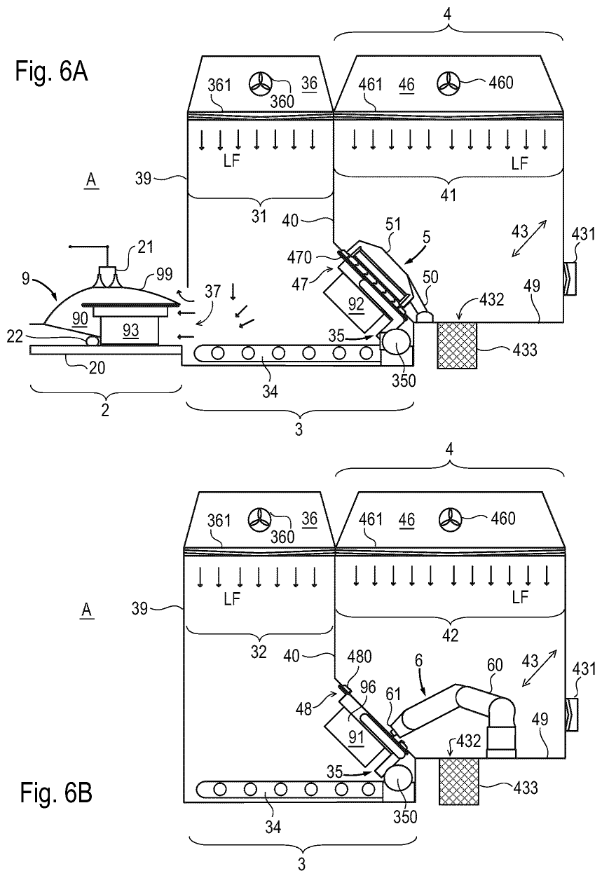

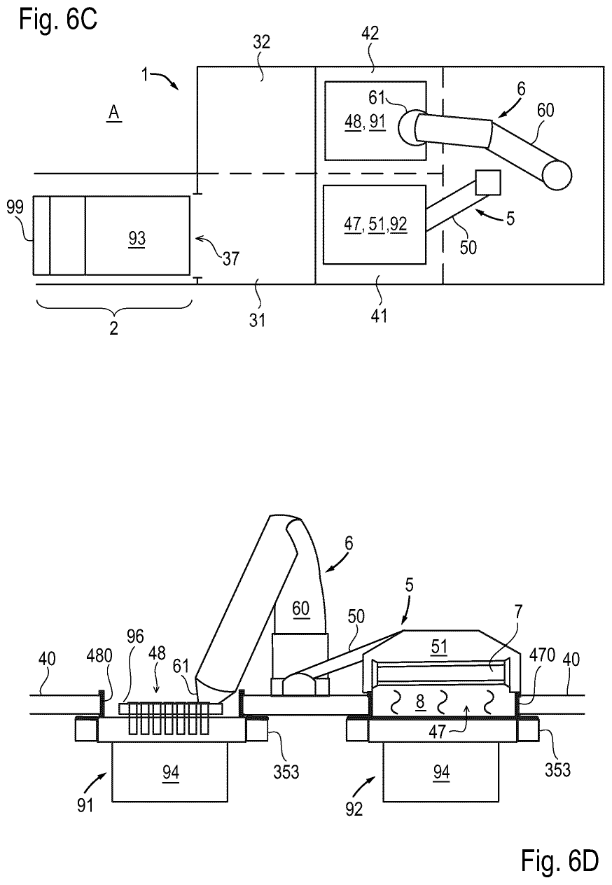

### Sequence in Process Phase 4 (FIGS. 6A to 6D)

In the providing station 2:

The new, third container 93 is pushed out of the enclosure 99, which is open on the side of the access point 37, by the feeding means 22, which is moved, toward the access point 37 in succession and in the process is irradiated by the cleaned flow LF. This prevents a lot of less-clean ambient air from the setup area A flowing around the container 93. The enclosure 99 is retained by the gripper 21.
In the containment 4, front first zone 41:
The decontamination device 7 is activated in order to decontaminate the gas volume present between the lid 51, the cover 98 on the first container 91, the seal 470 bordering the first passage 47 and possible exposed wall surfaces in the surrounding area 8, and all surfaces around which said gas volume flows.
In the containment 4, rear second zone 42:
A tool guided into abutment by the manipulator 6 transfers, for further processing, the nest 96, together with the articles 960 stored therein or only the articles 960, from the internal volume 90 of the vessel 94 of the first container 91 into the process chamber 43.

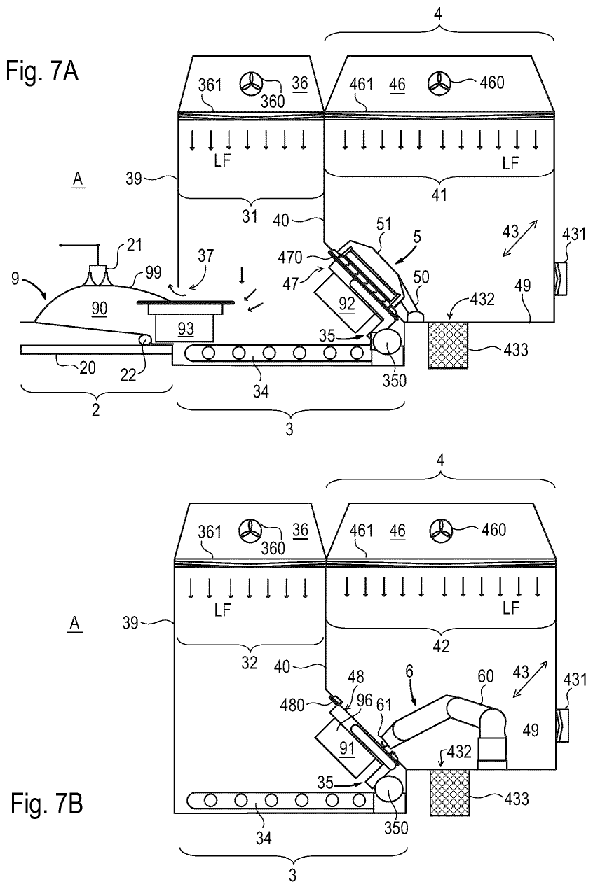

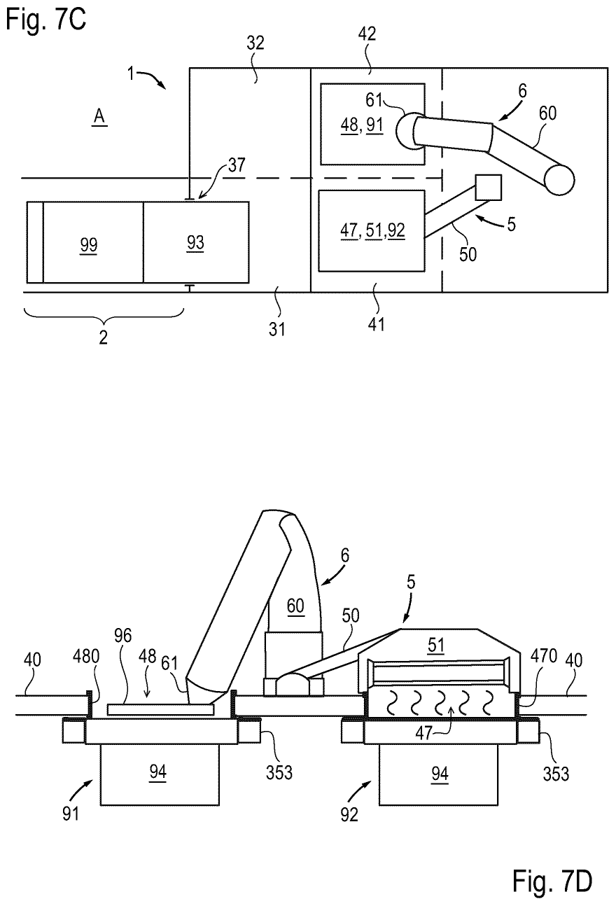

### Sequence in Process Phase 5 (FIGS. 7A to 7D)

In the providing station 2 and input station 3, front first zone 31:
The new, third container 93 is pushed by way of a pushing movement of the feeding means 22 out of the enclosure 99, through the access point 37 in the housing 39 and counter to the cleaned flow LF, onto the first conveying device 34 into the first zone 31 of the input station 3. The third container 93 is pushed out of the enclosure 99 directly onto the first conveying device 34, in order to avoid contact between the container 93 and the support 20 and thus not allow the transference of impurities. In the process, the gripper 21 retains the enclosure 99 for disposal purposes.
In the containment 4, rear second zone 42:
Into the vessel 94, which was emptied in a previous process phase, a nest 96 coming from the process chamber 43 in an earlier or the present production cycle is returned by means of the manipulator 6 to this vessel 94.

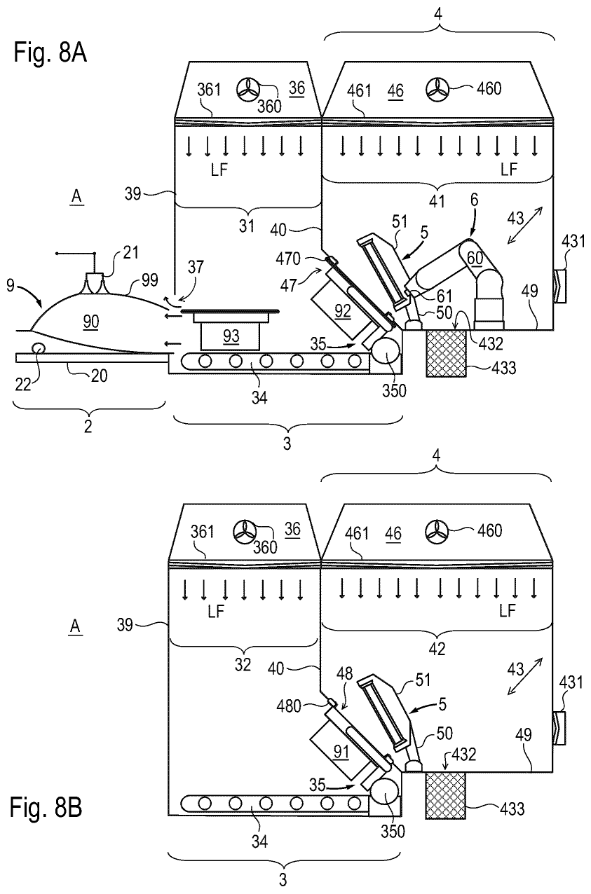

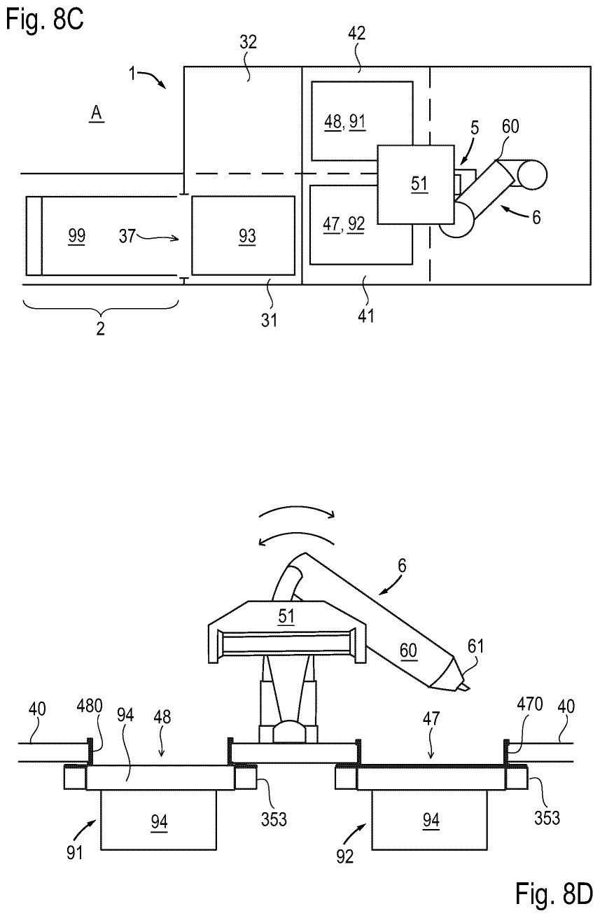

### Sequence in Process Phase 6 (FIGS. 8A to 8D)

In the providing station 2 and input station 3, front first zone 31:
The new, third container 93 is pushed completely into the first zone 31 and is carried by the first conveying device 34. The enclosure 99, which is retained by the gripper 21 and intended for disposal, and the feeding means 22, which have been moved back into the initial position, remain on the support 20.
In the containment 4, front first zone 41:
The decontamination device 7 integrated in the lid 51 can be switched off; the treatment has concluded. Pivoting the extension arm 50 of the apparatus 5 away causes the lid 51 to be lifted off of the first passage 47. At the same time, the tool head 61 of the manipulator 6 is moved back toward the first passage 47.
In the containment 4, rear second zone 42:
Pivoting the extension arm 50 of the apparatus 5 round causes the lid 51 to be brought up to the second passage 48.

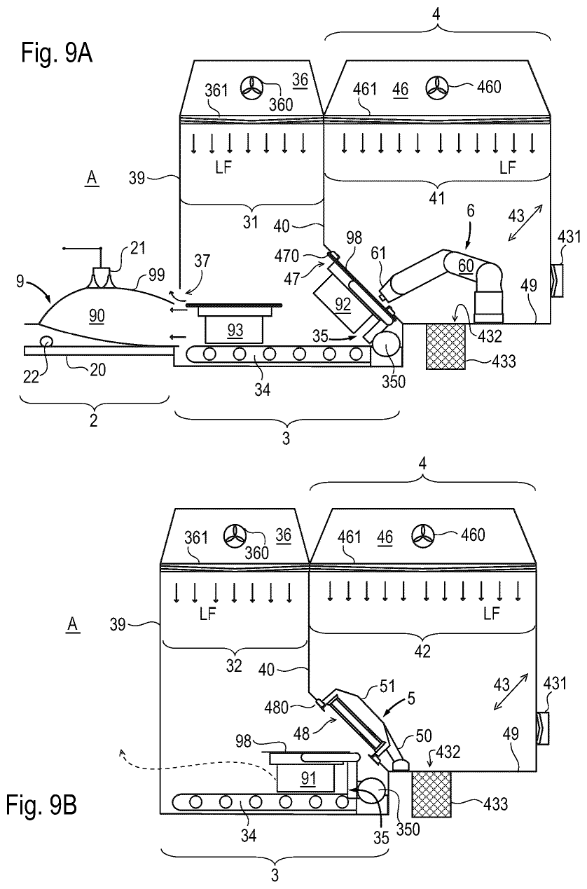

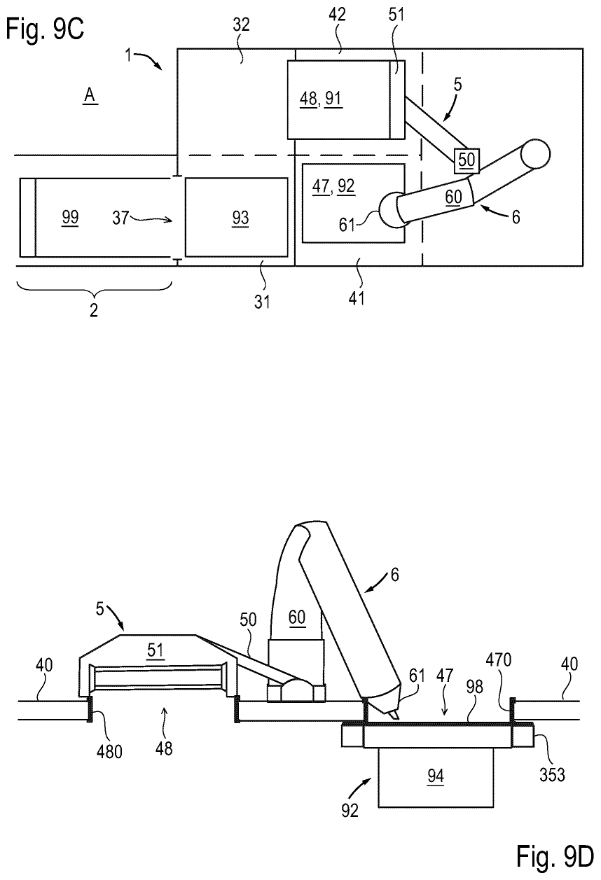

### Sequence in Process Phase 7 (FIGS. 9A to 9D)

In the containment 4, front first zone 41:
The lid 51 has been removed from the first passage 47 and the tool guided by the manipulator 6 is brought up to the cover 98 of the second container 92.
In the containment 4, rear second zone 42 and input station 3, rear second zone 32:
Pivoting the extension arm 50 of the apparatus 5 further brings the lid 51 with the decontamination device 7 integrated therein onto the second passage 48. Then, by pivoting the second conveying device 35 away, the vessel 94 of the processed first container 91 is removed from the second passage 48 and placed onto the first conveying device 34 for transport away outside of the lock arrangement 1.

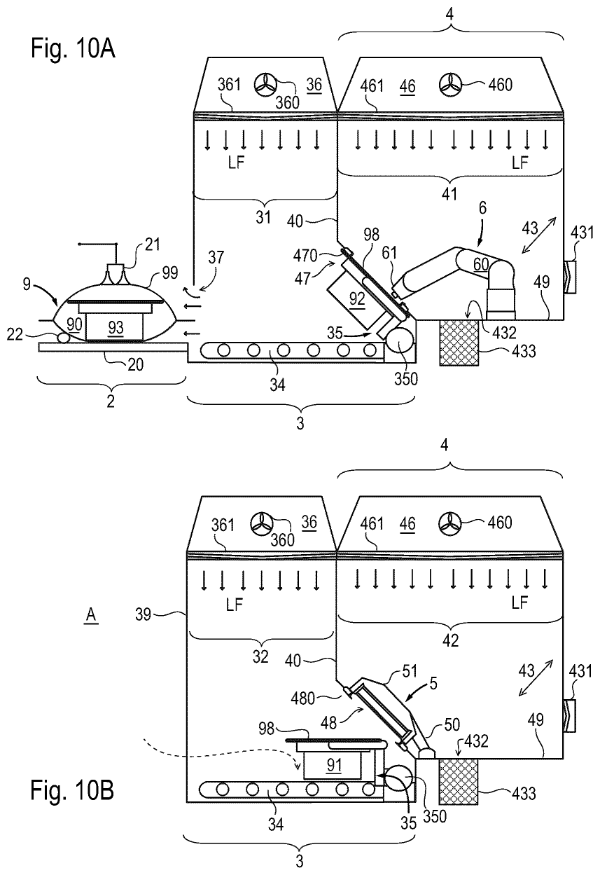

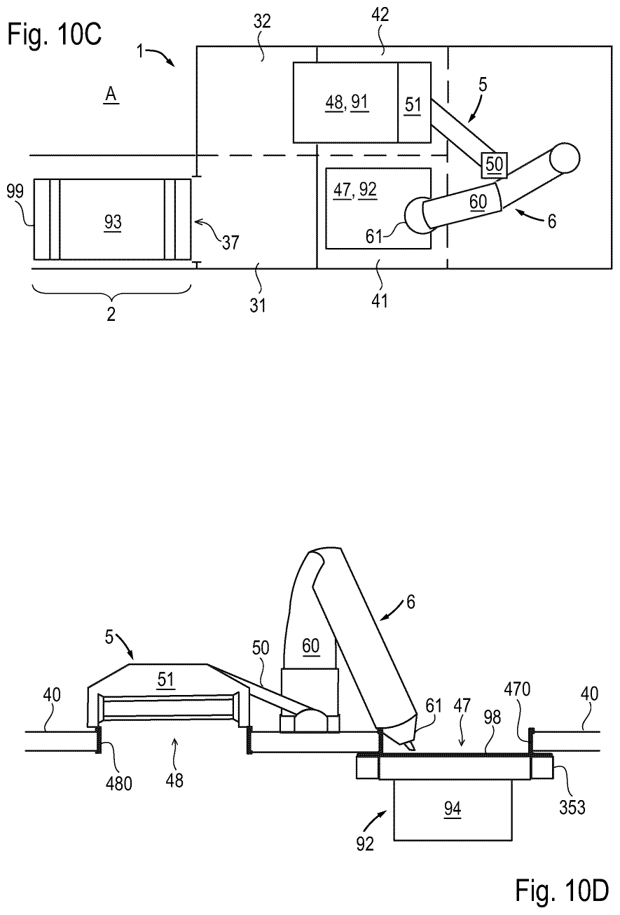

### Sequence in Process Phase 8 (FIGS. 10A to 10D)

In the providing station 2:
   Like in process phase 1 as per the start position, a next new, third container 93, in turn with the enclosure 99 still closed, is positioned on the support 20, in front of the access point 37.
   In the containment 4, front first zone 41:
   By means of the tool installed on the tool head 61 of the manipulator 6, the cover 98 of the second container 92, which is pressed against the first passage 47, is opened, e.g. by cutting it open, and the resulting cut waste is removed, with the result that access to the aperture 95 in the vessel 94 is provided.
In the input station 3, rear second zone 32:
A further first container 91, which has been freed of the enclosure 99 on the providing station 2 and has an intact cover 98, was transferred transversely onto the first conveying device 34 and in the meantime also loaded onto the second conveying device 35.

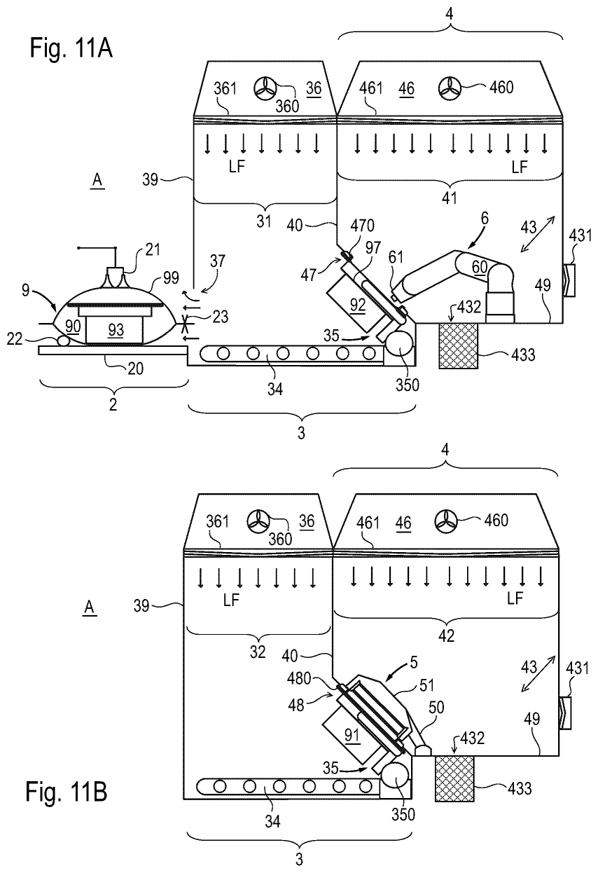

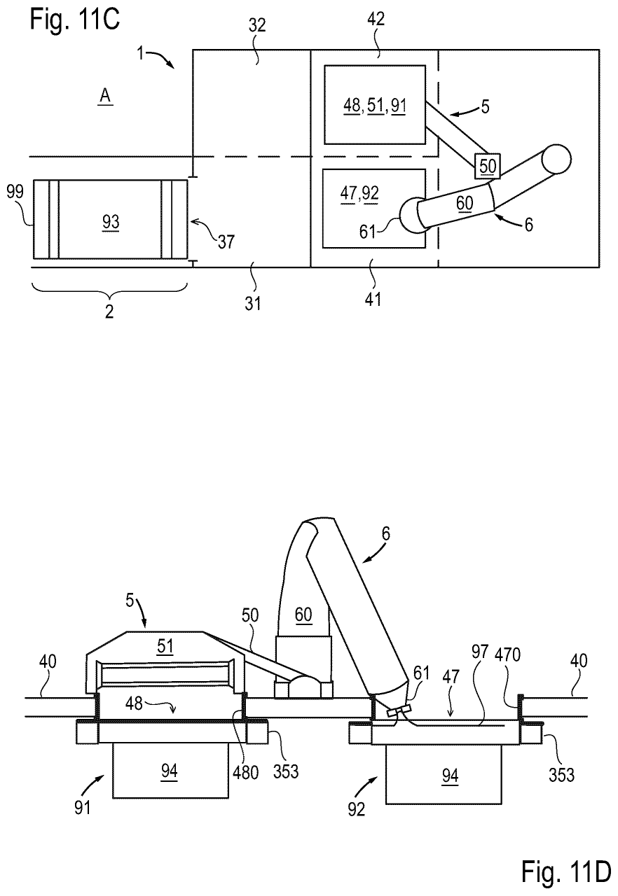

### Sequence in Process Phase 9 (FIGS. 11A to 11D)

In the providing station 2:
Like in process phase 3 as per FIG. 5A, the tool 23 is used to open the until-now closed enclosure 99.
In the containment 4, front first zone 41:
Like in process phase 3 as per FIG. 5B, the tool guided by the manipulator 6 is used, when the cover 98 is open, to remove the optionally present interlayer 97 from the second container 92, which is pressed against the first passage 47.
In the input station 3, rear second zone 32:
Pivoting the second conveying device 35 up causes the cover 98 of the further first, transversely transferred container 91 to be pressed against the second passage 48.
Therefore, as was already the case in process phase 3 as per FIG. 5A, recreated at the first passage 47, and now formed at the second passage 48, is a surrounding area 8 between the lid 51, the cover 98 of the second container 92, the seal 480 which borders the second passage 48, and possible exposed wall surfaces, including a gas volume that is enclosed by said surrounding area and is to be decontaminated in the next process phase, phase 10.

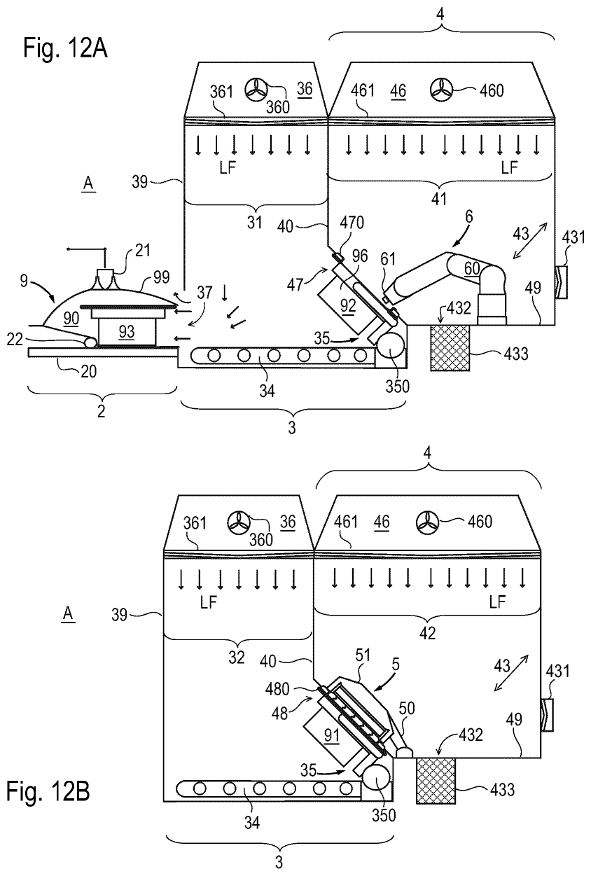

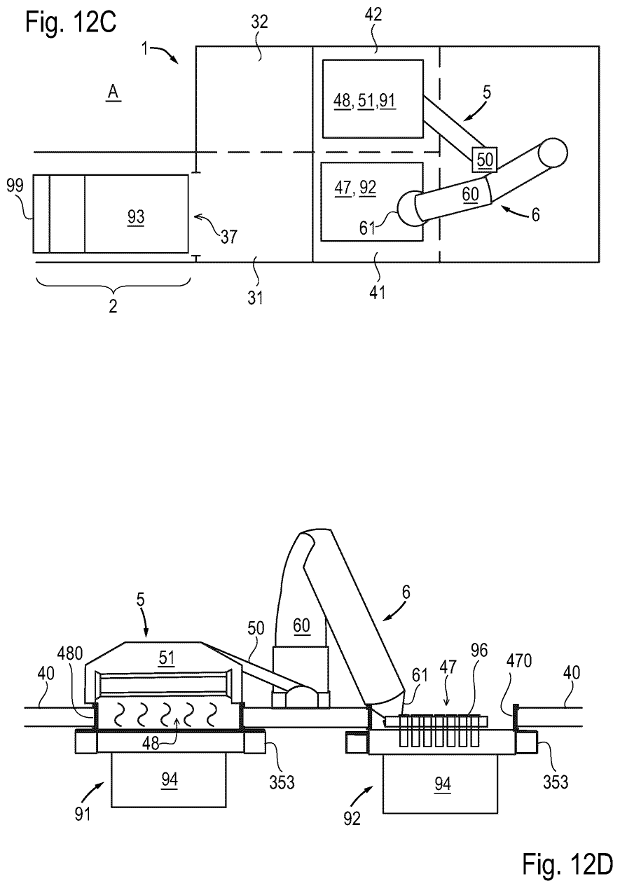

### Sequence in Process Phase 10 (FIGS. 12A to 12D)

In the providing station 2:
Here, the situation from process phase 4 as per FIG. 6A is repeated, but now with the next new, third container 93.
In the containment 4, front first zone 41:
Here, the situation from process phase 4 as per FIG. 6B is repeated, but this time with the second container 92, which is now pressed against the first passage 47.
In the input station 3, rear second zone 32, and containment 4, rear second zone 42:
The surrounding area 8, which was formed in the previous process phase, phase 9, as per FIG. 11B and encloses the gas volume, is decontaminated by means of the active decontamination device 7.

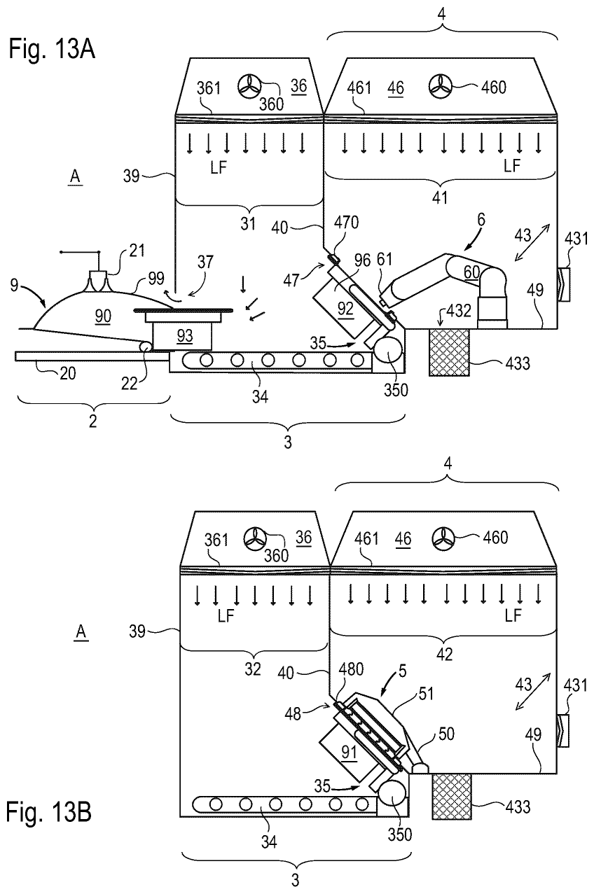

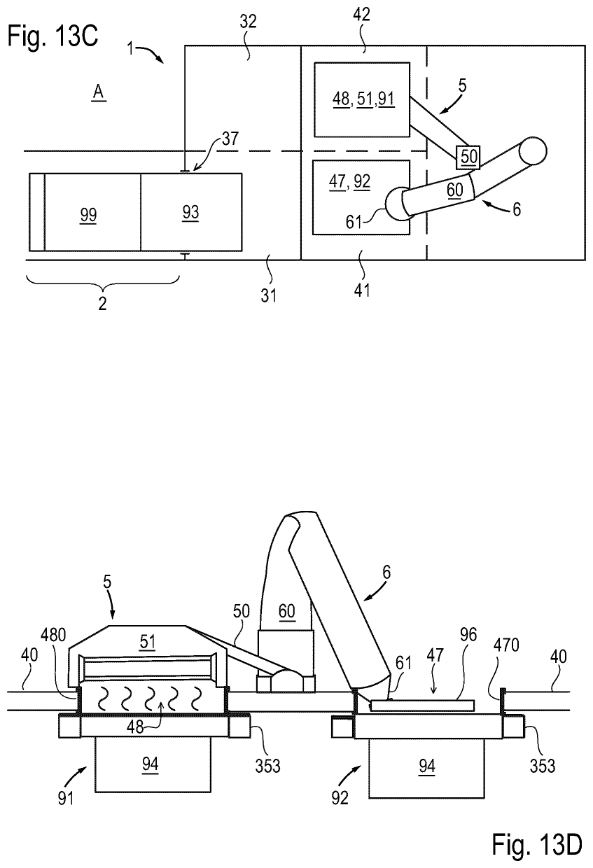

### Sequence in Process Phase 11 (FIGS. 13A to 13D)

In the providing station 2:
Here, the situation from process phase 5 as per FIG. 7A is repeated, but now with the next new, third container 93.
In the containment 4, front first zone 41:
Here, the situation from process phase 5 as per FIG. 7B is repeated, but this time an empty nest 96 from the process chamber 43 is returned to the vessel 94 of the second container 92, which is now pressed against the first passage 47.
In the input station 3, rear second zone 32, and containment 4, rear second zone 42:
The decontamination continues.

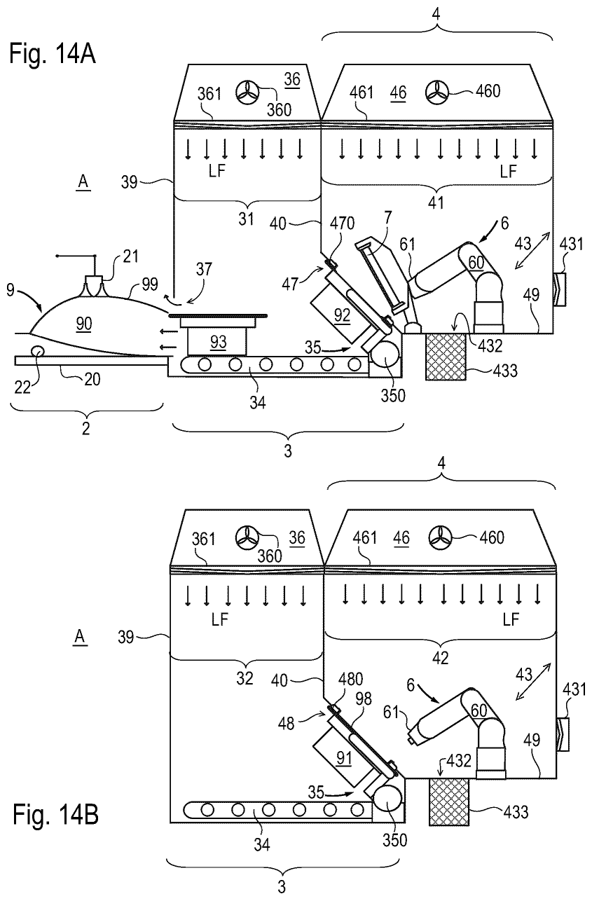

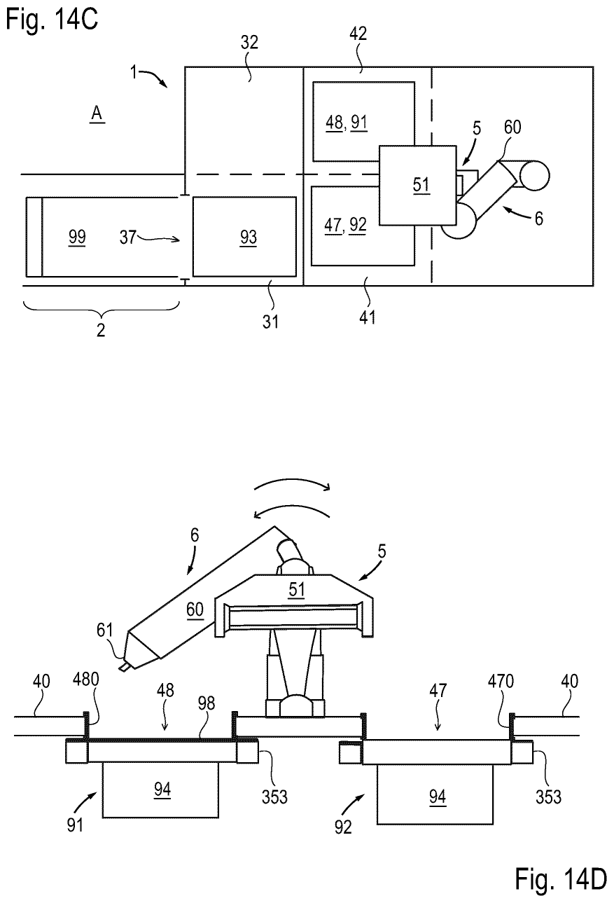

### Sequence in Process Phase 12 (FIGS. 14A to 14D)

In the providing station 2:
Here, the situation from process phase 6 as per FIG. 8A is repeated, but now with the next new, third container 93.
In the containment 4, front first zone 41:
Here, the situation from process phase 6 as per FIG. 8B is repeated, but now with the second container 92 and at the first passage 47, up to which the lid 51 is brought by pivoting the extension arm 50 of the apparatus 5 round.
In the input station 3, rear second zone 32, and containment 4, rear second zone 42:
The decontamination in the surrounding area 8 at the second passage 48 with the further first, transversely transferred container 91 is ended. The lid 51 with the decontamination device 7 is pivoted away and the manipulator 6 with the tools installed on the tool head 61 is pivoted into abutment with the second passage 48.

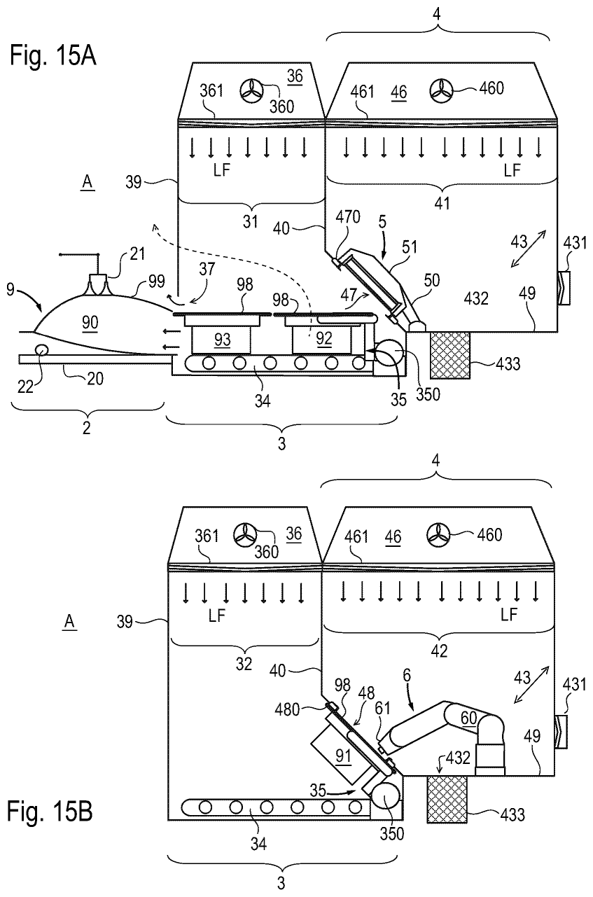

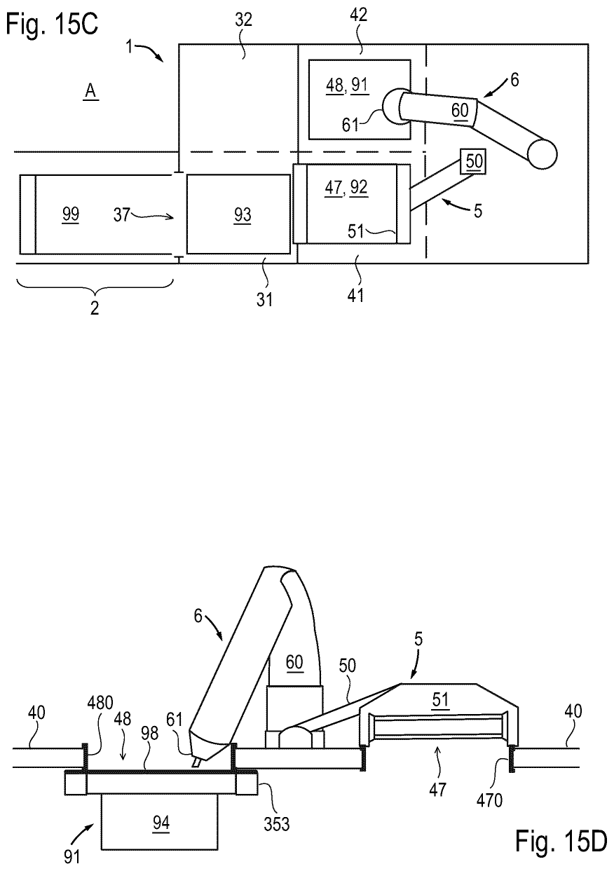

### Sequence in Process Phase 13 (FIGS. 15A to 15D)

In the input station 3, front first zone 31 and containment 4, front first zone 41:
First of all, the apparatus 5 pivots the lid 51 onto the first passage 47, closing it. Then, by pivoting the second conveying device 35 away, the vessel 94 of the processed second container 92 with the returned nest 96 is loaded onto the first conveying device 34 for transverse transport away outside of the lock arrangement 1. This avoids a collision with the next new, third container 93, which is already on the first conveying device 34.
In the input station 3, rear second zone 32 and containment 4, rear second zone 42:
Here, the situation as in process phase 1 as per FIG. 3B is repeated; a tool on the manipulator 6 is used to open the cover 98, but this time of the new first container 91 originally transferred transversely onto the first conveying device 34.

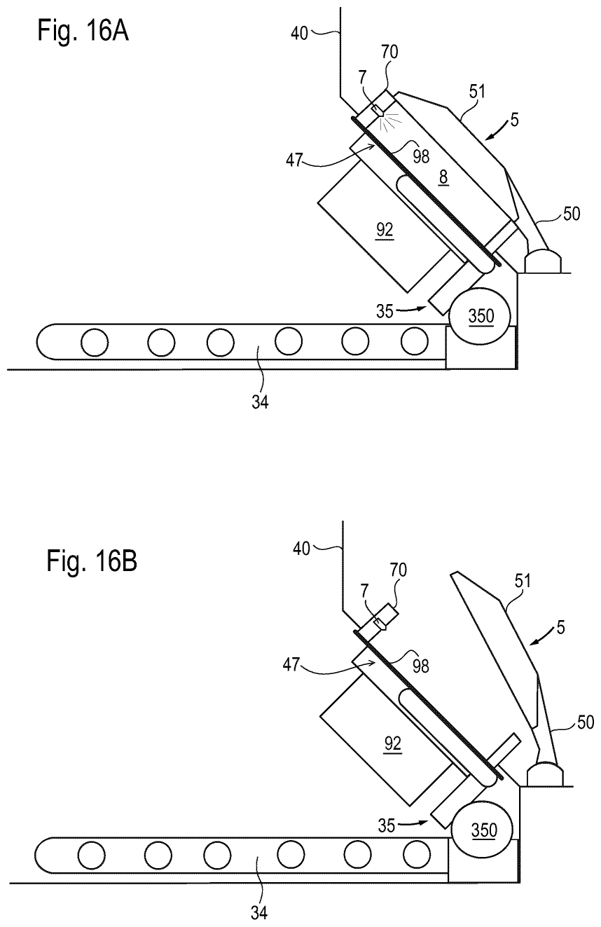

### FIGS. 16A and 16B

In this alternative arrangement, the decontamination device 7 is not, as before, integrated in the lid 51, but rather the emission element of the decontamination device 7, e.g. an atomizing nozzle for introducing an H₂O₂ aerosol, is not fitted in the lid 51, but rather outside in a mount 70. This mount 70 may be formed, very simply, by a chamfer of the partition wall 40 that extends between the input station 3 and the containment 4. The surface, facing toward the surrounding area 8, of the mount 70 is then a constituent part of the enclosed gas volume and surrounding area 8 to be decontaminated in the production process. The lid 51 is still carried by a pivotable extension arm 50 of an apparatus 5 stationed in the process chamber 43.

1. A method for the introduction of articles (960) under aseptic conditions from containers (9;91,92,93), which have been supplied in series, into a process chamber (43), which is surrounded by a housing (49), of a containment (4) by a lock arrangement (1) positioned in a setup area (1A), the method comprising:
a) providing an input station (3) upstream of the process chamber (43);
b) providing a single one of the containers (9;91,92,93) which comprises:
ba) a vessel (94) with an aseptic interior space (940) and an aperture (95), which has been closed with a cover (98); and
bb) the articles (960) stored in the interior space (940) which are to be treated in the process chamber (43) after the cover (98) is opened;
c) transferring the articles (960) from the containers (9;91,92,93) into the process chamber (43) from the input station (3) through at least two passages (47,48) leading into the process chamber (43), each of the passages (47,48) being closed on one side by a lid (51) that has been guided into abutment in the process chamber (43) and/or on an other side by the cover (98), facing the passage (47,48), of one said container (9;91,92,93) that has been guided into abutment in the input station (3);
d) when said container (9;91,92,93) has been guided into abutment with the passage (47,48), decontaminating that surface of the cover (98) that faces toward the passage (47,48), together with an enclosed surrounding area (8), using a decontamination device (7);
e) after that surface of the cover (98) that faces toward the passage (47,48), including the surrounding area (8) thereof, has been decontaminated, opening the cover (98) in order to transfer the articles (960) from the vessel (94) through the aperture (95) into the process chamber (43); and,
f) after the vessel (94) is emptied, firstly placing the lid (51) back onto the respective passage (47,48) in order to then remove the vessel (94), from which the articles (960) have been discharged, from the passage (47,48); and
g) alternately using the lid (51) on the passages (47,48).

2. The method as claimed in claim 1, wherein
a) the decontamination device (7) is integrated in the lid (51) or arranged above the lid (51); and
b) an emission element of the decontamination device (7) is arranged directly in the lid (51) or outside of the lid (51) in a receptacle (70).

3. The method as claimed in claim 1, wherein the enclosed surrounding area (8) includes a gas volume and surfaces around which the gas volume flows of at least one:
a) the lid (51);
b) the optional receptacle (70);
c) a seal (470,480); or
d) exposed wall areas.

4. The method as claimed in claim 1, wherein
a) the process chamber (43) is equipped with at least one manipulator (6) having pivotable extension arms (60) fitted in each case with a tool head provided with tools (61), wherein:
b) the tools are designed to open the cover (98) and then transfer the articles (960) from the vessel (94) through the opened-up aperture (95) into the process chamber (43); and
c) the at least one manipulator (6) with the tools is used interchangeably on the passages (47,48) to open the cover (98) and transfer the articles (960) into the process chamber (43); or
d) the at least one manipulator (6) includes first and second manipulators (6) that are each used only on one of the passages (47,48), or the first and second manipulators (6) are used interchangeably on the passages (47,48).

5. The method as claimed in claim 4, wherein
a) the tools include a cutting tool, and the method further comprises cutting open the cover (98) for the opening operation; wherein
b) at least parts of the area of the cover (98) and of the enclosure (99) consist of a semipermeable nonwoven fabric, and the cover (98), before it is removed, keeps the interior space (940) of the vessel (94) in a sterile state and is sealed on an edge (941) that runs around the aperture (95) of the vessel (94).

6. The method as claimed in claim 1, wherein
a) stored in the vessel (94) for receiving a multiplicity of articles (960) is a removable nest (96) which, together with the articles (960), is transferred into the process chamber (43); and,
b) after the articles (960) have been discharged from the nest (96) in the process chamber (43), the method further comprises clearing the empty nest (96) from the process chamber (43) into an empty vessel (94) which comes next or which is the same vessel and has been pressed against the respective passage (47,48); or
c) transferring the articles (960) directly from the nest (96) that remains in the vessel (94) into the process chamber (43).

7. The method as claimed in claim 1, wherein the decontamination device comprises a radiation source, E-beam or pulsed light, or a fumigation device.

8. The method as claimed in claim 1, wherein the input station (3) is surrounded by a housing (39), which has an access point (37) leading to an adjacent providing station (2), and the method further comprises:
a) operating the input station (3) with a cleaned, unidirectional displacement flow (LF), some of which exits through the access point (37);
b) at the providing station (2), supplying the containers (9;91,92,93) that, in the as-supplied state, are each surrounded by an enclosure (99) in order to keep the internal volume (90) of the container (9;91,92,93), together with the contents therein, aseptic; and
c) at the providing station (2), opening up an opening, which has been made on the enclosure (99) and until now was kept closed, on a side facing toward the displacement flow (LF) exiting the access point (37), in order to introduce the single container (9;91,92,93) into the input station (3); or
d) at the providing station (2), separating the enclosure (99) on the side facing toward the displacement flow (LF) exiting the access point (37), in order to introduce the single container (9;91,92,93) into the input station (3).

9. The method as claimed in claim 8, further comprising
a) positioning the container (9;91,92,93) fitted in the initially still closed enclosure (99) on a support (20);
b) with a feeding means (22), pushing the container (9;91,92,93) out of the open enclosure (99), when there is a displacement flow (LF) flowing onto the container (9;91,92,93); and
c) holding the enclosure (99) by a gripper (21).

10. The method as claimed in claim 1, further comprising:
a) feeding the containers (9;91,92,93) that have been introduced into the input station (3) to the passages (47,48) by at least one conveying device (34,35); and
b) wherein the at least one conveying device (34,35) is a magnetic levitation conveying device.

11. The method as claimed in claim 10, wherein the at least one conveying device comprises first and second conveying devices, and the method further includes:
a) using the first conveying device (34) to transport the containers (9;91,92,93) within the input station (3);
b) the second conveying device (35) pivoting the containers (9;91,92,93) to be pivoted into abutment with the respective cover (98) pressed against the respective passage (47,48); and
c) using the second conveying device (35) to remove processed containers (9;91,92,93) from the respective passage (47,48).

12. The method as claimed in claim 11, wherein, for positioning the containers (9;91,92,93) against the respective passage (47,48), the second conveying device (35) comprises a cardanic bearing (351).

13. The method as claimed in claim 12 the second conveying device (35) comprises:
a) a shaft (350), through which a first axis of rotation (D1) extends;
b) two forks (352), which are fixed next to one another on the shaft (350) and are each pivotable about a second axis of rotation (D2) extending at right angles to the first axis of rotation (D1); and
c) a respective carrier (353) which is suspended in the associated fork (352) and is pivotable about a third axis of rotation (D3), wherein the first axis of rotation (D1) and the third axis of rotation (D3) run parallel to one another and the carriers (353) are configured to grip edges (941) of the vessels (94) from underneath while the vessels are being pivoted onto and off of the passages (47,48); wherein
d) two of the cardanic bearings (351) that are next to one another are each formed from a U-shaped carrier (353), which is connected in the third axis of rotation (D3) in each case to one said fork (352), which is suspended in the second axis of rotation (D2).

14. The method as claimed in claim 1, wherein a seal is fitted in each said passage (47,48), the seal (470,480) which, together with the cover (98) of the container (9;91,92,93) which has been temporarily pressed against and/or together with the lid (51) which has been temporarily pressed against the passages (47,48), provides sealing in the transition from the input station (3) to the process chamber (43).

15. The method as claimed in claim 1, wherein the lid (51) is carried by a pivotable extension arm (50) of an apparatus (5) positioned in the process chamber (43).

16. The method as claimed in claim 4, wherein the at least one manipulator (6) comprises a robot.

17. The method as claimed in claim 1, wherein the vessels (94) of the containers (9;91,92,93) comprise trough-shaped tubs and the articles (960) are at least one of ampullae, vials or syringes which fit into systematically arranged receiving contours of nests (96) in the containers.

18. A lock arrangement for the introduction of articles (960) under aseptic conditions from containers (9;91,92,93), which have been supplied in series, into a process chamber (43), which is surrounded by a housing (49), of a containment (4) a via the lock arrangement (1) positioned in a setup area (A), the lock arrangement comprising:
a) an input station (3) upstream of the process chamber (43);
b) wherein a single one of the containers (9;91,92,93) comprises:
ba) a vessel (94) with an aseptic interior space (940) and an aperture (95), which has been closed with a cover (98); and
bb) the articles (960) stored in the interior space (940) that are to be treated in the process chamber (43) after the cover (98) is opened;
c) at least two passages (47,48) leading into the process chamber (43) from the input station (3) to transfer the articles (960) from the containers (9;91,92,93) into the process chamber (43), each of the passages (47,48) is closeable on one side by a lid (51) that has been guided into abutment in the process chamber (43) and/or on an other side by the cover (98), facing the passage (47,48), of the respective container (9;91,92,93) that has been guided into abutment with the input station (3);
d) a decontamination device (7), wherein the container (9;91,92,93) guided into abutment with the passage (47,48), has a surface of the cover (98) that faces toward the passage (47,48), together with an enclosed surrounding area (8), which is adapted to be decontaminated by the decontamination device (7);
e) wherein after that surface of the cover (98) that faces toward the passage (47,48), including the surrounding area (8) thereof, has been decontaminated, the cover (98) is adapted to be opened and the articles (960) are adapted to be transferred from the vessel (94) through the aperture (95) thereof into the process chamber (43); and
f) the lid (51) is adapted to be connected back onto the respective passage (47,48), which is the one the empty vessel (94) abuts, and thus the empty vessel (94) is removable from the passage (47,48); wherein
g) the lid (51) is used alternately between the passages (47,48).

19. The lock arrangement as claimed in claim 18, wherein
a) the decontamination device (7) is integrated in the lid (51) or arranged above the lid (51); and
b) an emission element of the decontamination device (7) is arranged directly in the lid (51) or outside of the lid (51) in a receptacle (70).

20. The lock arrangement as claimed in claim 18, wherein the enclosed surrounding area (8) includes a gas volume and surfaces around which the gas volume flows, including surfaces of at least one of
a) the lid (51);
b) the optional receptacle (70);
c) a seal (470,480); or
d) exposed wall areas.

21. The lock arrangement as claimed in claim 18, wherein
a) the process chamber (43) is equipped with at least one manipulator (6) having pivotable extension arms (60) with in each case a tool head provided with tools (61) fitted thereon, wherein:
b) the tools are designed to open the cover (98) and then transfer the articles (960) from the vessel (94) through the opened-up aperture (95) into the process chamber (43); and
c) the at least one manipulator (6) with the tools is used interchangeably on the passages (47,48) to open the cover (98) and transfer the articles (960) into the process chamber (43); or
d) the at least one manipulator (6) includes first and second manipulators (6) each of which is used only on one of the passages (47,48), or the the first and second manipulators (6) are used interchangeably on the passages (47,48).

22. The lock arrangement as claimed in claim 21, wherein
a) the tools include a cutting tool intended for opening the cover (98); wherein
b) at least parts of the area of the cover (98) and of the enclosure (99) consist of a semipermeable nonwoven fabric, and the cover (98), before it is removed, keeps the interior space (940) of the vessel (94) in a sterile state and is sealed on an edge (941) that runs around the aperture (95) of the vessel (94).

23. The lock arrangement as claimed in claim 18, further comprising
a) a removable nest stored in the vessel (94) for receiving a multiplicity of articles (960), the removable nest (96), together with the articles (960), is adapted to be transferred into the process chamber (43); and
b) the nest (96) from which articles (960) have been discharged is adapted to be cleared from the process chamber (43) into an empty vessel (94) which comes next or which is the same one and has been docked at the respective passage (47,48); or
c) the articles (960) are adapted to be transferred directly from the nest (96) that remains in the vessel (94) into the process chamber (43).

24. The lock arrangement as claimed in claim 18, wherein the decontamination device comprises a radiation source or a fumigation device.

25. The lock arrangement as claimed in claim 18, wherein the input station (3) is surrounded by a housing (39), and the housing has an access point (37) leading to an adjacent providing station (2), wherein:
a) the input station (3) is adapted to be operated with a cleaned, unidirectional displacement flow (LF), some of which exits through the access point (37);
b) the providing station (2) is adapted to supply containers (9;91,92,93) that, in the as-supplied state, are each surrounded by an enclosure (99) in order to keep an internal volume (90) of the container (9;91,92,93), together with the contents therein, aseptic; and
c) at the providing station (2), an opening, which has been made on the enclosure (99) and until now was kept closed by a placed-on tool, is adapted to then be opened up, by releasing the tool, on the side facing toward the displacement flow (LF) exiting the access point (37), with the result that the single container (9;91,92,93) is introducible into the input station (3); or
d) at the providing station (2), the enclosure (99) is adapted to be separated by a tool on the side facing toward the displacement flow (LF) exiting the access point (37), with the result that the single container (9;91,92,93) is introducible into the input station (3).

26. The lock arrangement as claimed in claim 25, further comprising:
a) a support (20) for positioning the container (9;91,92,93) fitted in the initially still closed enclosure (99);
b) a feeding means (22) to push the container (9;91,92,93) out of the open enclosure (99), when there is a displacement flow (LF) flowing onto the container (9;91,92,93); and
c) a gripper (21) to retain the enclosure (99).

27. The lock arrangement as claimed in claim 18, further comprising:
a) at least one conveying device (34,35) to feed the containers (9;91,92,93) introduced into the input station (3) to the passages (47,48); and
b) the at least one conveying device (34,35) is a magnetic levitation conveying device.

28. The lock arrangement as claimed in claim 27, wherein the at least one conveying device includes
a) a first conveying device (34) to transport the containers (9;91,92,93) within the input station (3); and
b) a second conveying device (35) that pivots the containers (9;91,92,93) so that the cover (98) is pressed against the respective passage (47,48); and
c) the second conveying device (35) is adapted to remove processed containers (9;91,92,93) from the respective passage (47,48).

29. The lock arrangement as claimed in claim 28, wherein, to position the containers (9;91,92,93) against the respective passage (47,48), the second conveying device (35) comprises a cardanic bearing (351).

30. The lock arrangement as claimed in claim 29, wherein the second conveying device (35) comprises:
a) a shaft (350), through which a first axis of rotation (D1) extends;
b) two forks (352), which are fixed next to one another on the shaft (350) and are each pivotable about a second axis of rotation (D2) extending at right angles to the first axis of rotation (D1); and
c) a respective carrier (353) which is suspended in the associated fork (352) and is adapted to be pivoted about a third axis of rotation (D3), wherein the first axis of rotation (D1) and the third axis of rotation (D3) run parallel to one another and the carriers (353) are configured to grip edges (941) of the vessels (94) from underneath while the vessels are being pivoted onto and off of the passages (47,48); and
d) there are two of the cardanic bearings (351) that are next to one another and are each formed from a U-shaped carrier (353), which is connected in the third axis of rotation (D3) in each case to one of the forks (352), which is suspended in the second axis of rotation (D2).

31. The lock arrangement as claimed in claim 18, wherein fitted in each said passage (47,48) is a seal (470,480) which, together with the cover (98) of the container (9;91,92,93) which has been temporarily pressed against and/or together with the lid (51) which has been temporarily pressed against the passages (47,48), provides sealing in a transition from the input station (3) to the process chamber (43).

32. The lock arrangement as claimed in claim 18, further comprising a pivotable extension arm (50) of an apparatus (5) positioned in the process chamber (43) that carries the lid (51).

33. The lock arrangement as claimed in claim 21, wherein the at least one manipulator (6) comprises a robot.

34. The lock arrangement as claimed in claim 18, wherein the vessels (94) of the containers (9;91,92,93) have trough-shaped tubs and the articles (960) are at least one of ampullae, vials or syringes which fit into systematically arranged receiving contours of nests (96) in the containers.
